(19) United States
(12) Patent Application Publication
(10) Pub. No.: US 2006/0173261 A1
(43) Pub. Date: Aug. 3, 2006

(54) Biopotential sensor

(76) Inventors: Magnus Kall, Helsinki (FI); Edvard Kalvesten, Stockholm (SE)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/052,740

(22) Filed: Jan. 31, 2005

(51) Int. Cl. A61B 5/04

(52) U.S. Cl. 600/372

(57) ABSTRACT

A sensor for measuring biopotential signals from the skin of a patient. The sensor comprises a carrier having a top surface positioned contiguous with the skin of the patient when the sensor is applied to the patient's skin and a bottom surface facing away from the patient when the sensor is applied to the patient's skin. The carrier has further plurality of spikes extending from the top surface for being inserted into the skin of the patient when the sensor is applied to the patient's skin. The sensor further comprises means for obtaining the biopotential signal and for making the biopotential signal available externally of the sensor. The carrier is manufactured of non-conductive material and the non-conductive material is made conductive to form electrical connection between the top and bottom surfaces of the carrier.

### BACKGROUND AND SUMMARY

The invention relates to medical sensors, which are applied to a patient's skin for monitoring biopotentials and in particular to sensors that penetrate the patient's skin to make the skin more electrically permeable.

Diagnostic tests, treatments and the presence of illness require measuring and monitoring electrical signals generated by the physiological functioning of a patient. Typical electrical signals or biopotentials that are commonly monitored are those producing electrocardiograms (ECG) from the heart, electroencephalograms (EEG) from the brain and electromylograms (EMG) from the muscles. Such signals are of relatively low level and may be weak, such as 100 microvolt or less signals present in an electroencephalogram (EEG). The frequency range of the signals extends from 0.05 for electro-cardiograms to 3000 Hz for brain stem evoked potentials.

Skin mounted monitoring sensors are typically used to obtain the biopotentials. The human skin is made of three distinct layers; stratum corneum, viable epidermis, and dermis. The outer 10-15 micrometers of skin, called the stratum corneum, is dead tissue that forms the primary barrier for the body. The stratum corneum is the major contributor to skin impedance and to a reduction in biopotential signal magnitudes as well as a major factor in the signal to noise ratio characteristics of skin-mounted sensors. Below the stratum corneum lies the viable epidermis (50-100 micrometers). The viable epidermis is comprised of living cells, but contains few nerves and is devoid of blood vessels. Penetration of the skin to the viable epidermis is painless since the nerves are found in deeper tissues. Below the viable epidermis is the dermis. The dermis forms the bulk of skin volume and contains living cells, nerves and blood vessels.

Difficulties often arise when measuring weak biopotentials with skin mounted electrodes or sensors. One problem is that the outermost layer of skin has high electrical impedance. High electrical impedance reduces signal magnitude so that a data signal may be difficult to obtain when electrical noise is present.

When measuring biopotentials with a sensor placed on the patient's skin, it is of primary interest to obtain as low contact impedance as possible, i.e. it is a common practice to remove a part of the stratum corneum prior to applying the sensor so as to lessen the skin impedance. Traditionally this has involved rigorous preparation of the skin at the location where the biopotential sensor is applied. This rigorous preparation includes removal of grease and oils by means wiping with e.g. alcohol as well as removal of dead, insulating skin cells by means of abrasion. After the biopotential sensor is placed on the prepared area there is typically a stabilizing period before a reliable signal is obtained.

The procedure described above is however time consuming, particularly when several sensors are to be applied and is inconvenient in any clinical situations such as preparing the patient for surgery.

In order to eliminate the disadvantages described above piercing of the skin to reduce its impedance has been suggested. A device, i.e a sensor for doing this comprises spikes penetrating the dead, insulating outer layer of the skin into the conductive part of the skin, however not penetrating as deep as to come in contact with blood circulation and nerve cells. This allows for quick application without any rigorous skin preparation typically required with traditional biopotential sensors to obtain a good skin contact. This sensor also gives a good contact at initial application of the sensor and does not need as long a stabilization time as a traditional biopotential sensor. As an example of these skin-piercing biopotential sensors can be mentioned the one described in U.S. Pat. No. 6,622,035.

The object of the invention is to obtain a simple and practical structure for a biopotential sensor. An advantage of the present invention is that the sensor is extremely simple and practical when compared to the prior art. Simple structure means in practice that the invention can be materialized very simply, whereby costs can be kept at reasonably low level.

### BRIEF DESCRIPTION OF THE DRAWING

In the following the invention will be described by means of the examples shown in the drawing, in which

FIG. 1 shows schematically the basic steps of the manufacture of the sensor of the invention,

FIG. 2 shows a perspective view of the sensor manufactured along the teachings of FIG. 1,

FIG. 3 shows schematically a partial section view of the sensor of the invention showing spikes embedded into the skin, and

FIG. 4 shows schematically a partial section view of an alternative embodiment of the sensor of the invention.

### DETAILED DESCRIPTION

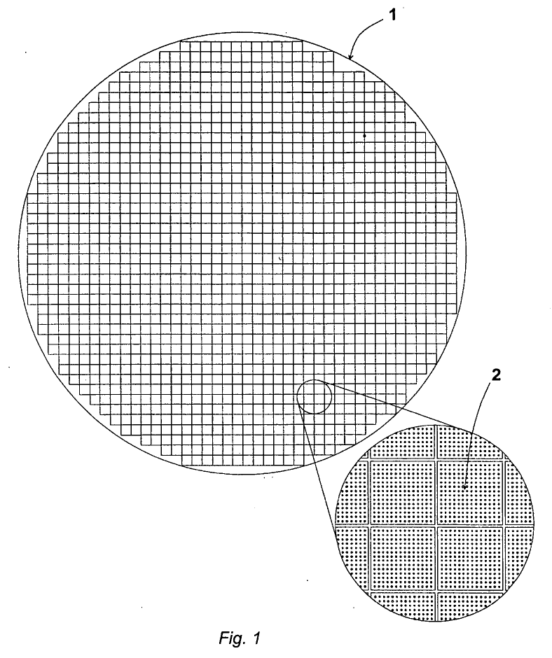

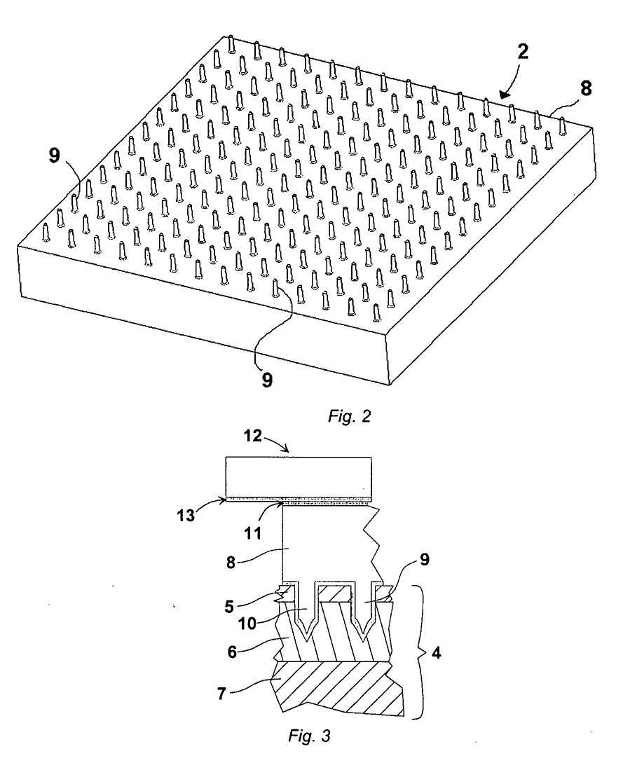

FIG. 1 shows basic steps of the manufacture of the sensor of the invention. Micromechanic structures are manufactured by etching a material, for example silicon, into the desired shape. This allows the manufacturing of complex structures with extremely small dimensions. The spikes in the biopotential sensor of the invention are etched on a silicon wafer 1, which is thereafter coated with conductive substances to make a suitable skin interface. In order to avoid having to coat each sensor separately, the entire wafer is coated at once. After the coating steps the wafer 1 is diced into sensors 2. This results in a “cube” having a carrier 8 and spikes 9 extending from the top surface of the carrier. The top surface of the carrier and the surfaces of the spikes are coated with a conductive material but the sides are bare Si. Said “cube” formed sensor structure is clearly shown in FIG. 2. The upper edges can be rounded or chamfered to make the coatings reach further down the sides of the sensor. The bottom surface of the wafer can be coated in a similar fashion, to ease the attachment of the sensor to an assembly.

FIG. 3 shows a schematic representation of the sensor 2 of the invention applied to the skin 4 of the patient. As told above skin 4 is formed of three layers, stratum corneum 5, viable epidermis 6 and dermis 7. Stratum corneum 5 is 10-15 micrometers thick and consists of dead tissue. Underneath stratum corneum 5 is viable epidermis 6. Viable epidermis 6 is 50-100 micrometers thick and consists of tissue containing living cells. This tissue contains few nerves and is devoid of blood vessels. Below viable epidermis 6 is dermis 7. Dermis 7 comprises living cells, nerves and blood vessels.

Preferably the spikes 9 projecting from the carrier 8 penetrate skin 4 so that spike tips lie within viable epidermis 6. This provides impedance reducing, electrical signal pathways across stratum corneum 5 without causing pain or contact to blood circulation of the patient. Reference number 10 shows the layer of electrically conducting material, for example Ag, applied onto the top surface of the carrier 8 and onto the surfaces of the spikes 9. Reference number 11 shows the layer of electrically conducting material, for example Ag, applied onto the bottom surface of the carrier 8.

A biopotential sensor transforms an ionic biopotential into an electronic potential, which is transferred to measuring electronics. In order to transfer the signal, the conductive coating on the sensor must be electrically connected to a conductor, which in turn transfers the signal to the measurement electronics. This can be achieved by creating an electrical connection from the skin 4 to the bottom surface of the carrier 8. As told above in the present invention the carrier and the spikes are made of silicon bulk material. The gist in the present invention is to make the otherwise non-conductive silicon bulk material conductive. According to the first embodiment of the invention silicon bulk material can be made conductive by doping the silicon material with impurities such as antimony, arsenic, phosphorus, boron, aluminium or gallium.

The process of doping introduces an atom of another element into the silicon crystal to alter its electrical properties. The “dopant”, which is the introduced element, has either three or five valence electrons, which is one less or one more that silicon's four. There are several methods of introducing impurities into the silicon. One method is Ion Implantation. This method bombards the wafer with the desired impurities by selecting the required impurities and speeding them up by giving them energy. Another method is to coat a layer of silicon material with impurities and then heat the surface. This allows the impurity atoms to diffuse into the silicon. The amount of impurities added is in the range of 0,1 . . . 100 parts per million atoms.

Creating this way electrical connection from the skin to the electrically conductive material layer 11 applied onto the bottom surface of the carrier offers a straightforward and cost-effective way to materialize a practical biopotential sensor. In this connection it is important to realize that signal can be transferred very simply from the conductive material layer 11 to the measurement electronics. The measuring electronics can be integrated into an assembly as schematically shown in FIG. 3. Said measuring electronics can be connected to the sensor by means of a conductor. By having the bottom surface of the carrier 8 conductive with the conductive material layer 11, the sensor can be automatically assembled if needed using standard electronics assembly tools. The sensor can automatically be picked from the polymer film on which it is manufactured and placed on an appropriate assembly 12 as shown in FIG. 3. Solder or conductive adhesive material can be used in forming the connection between the electrically conductive layer 11 and the co-operating connection layer 13 of the assembly 12.

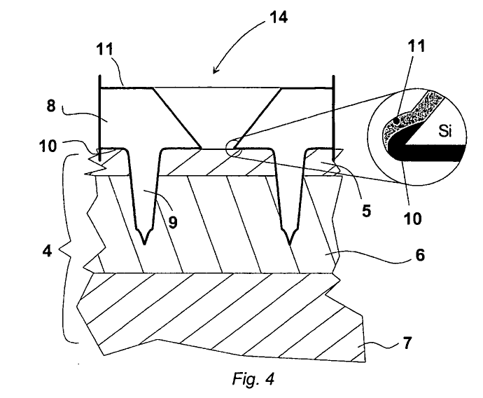

FIG. 4 shows an alternative embodiment of the invention. FIG. 4 shows this alternative embodiment in the same way as FIG. 3 shows the first embodiment. In this embodiment holes 14 are made through the wafer 1 in a pattern with a pitch smaller than the chip size. The holes 14 can be made by using any appropriate way, for example by etching. In this embodiment both sides of the chip are coated with conductive material, for example Ag, so that the conductive material overlaps at the holes to form the electrical connection between the top surface and the bottom surface of sensor. Said overlapping of the conductive material layers can be clearly seen in the part of FIG. 4 showing a detail of the embodiment in a greater scale. As shown in FIG. 4 the conductive material of the top and the bottom surfaces of the carrier 8 form electrical connection through the otherwise non-conductive bulk material. In this embodiment the non-conductive material can be for example silicon as described also in the embodiment of FIG. 3.

The connection between the assembly and the electrically conductive layer 11 can be formed in the same way as described in FIG. 3.

The embodiments described above are not intended to restrict the invention but only to clarify the basic idea of the invention. It is quite clear that details can be varied within the scope of the claims. The invention has been described above with an embodiment using silicon as a bulk material. The invention is however not restricted only to silicon but also other suitable materials, for example appropriate plastic materials can be used.

1. A sensor for measuring biopotential signals from the skin of a patient, the sensor comprising:
a carrier having a top surface positioned contiguous with the skin of the patient when the sensor is applied to the patient's skin and a bottom surface facing away from the patient when the sensor is applied to the patient's skin, the carrier further having plurality of spikes extending from the top surface for being inserted into the skin of the patient when the sensor is applied to the patient's skin,
means for obtaining the biopotential signal and for making the biopotential signal available externally of the sensor,
the carrier being manufactured of non-conductive material and the non-conductive material being doped with impurities to make it conductive to form electrical connection between the top and bottom surfaces of the carrier.

2. The sensor of claim 1 wherein the top surface of the carrier is coated with an electrically conductive material.

3. The sensor of claim 2 wherein the conductive coating material is silver.

4. The sensor of claim 1 wherein the bottom surface of the carrier is coated with an electrically conductive material.

5. The sensor of claim 4 wherein the conductive coating material is silver.

6. The sensor of claim 1 wherein the top surface and the bottom surface are coated with an electrically conductive material.

7. The sensor of claim 6 wherein the conductive coating material is silver.

8. The sensor of claim 1 wherein the carrier is made of silicon material.

9. The sensor of claim 1 wherein the amount of impurities is in the range of 0,1 . . . 100 parts per million atoms.

10. A sensor for measuring biopotential signals from the skin of a patient, the sensor comprising:
a carrier having a top surface positioned contiguous with the skin of the patient when the sensor is applied to the patient's skin and a bottom surface facing away from the patient when the sensor is applied to the patient's skin, the carrier further having plurality of spikes extending from the top surface for being inserted into the skin of the patient when the sensor is applied to the patient's skin, and the carrier being manufactured of non-conductive material,
means for obtaining the biopotential signal and for making the biopotential signal available externally of the sensor,
the carrier having holes made over the surfaces thereof through the non-conductive material, and the top surface and the bottom surface of the carrier being coated with an electrically conductive material, the electrically conductive material overlapping at he holes to form electrical connection between the top and the bottom surfaces of the carrier.

11. The sensor of claim 10 wherein the carrier is made of silicon material.

12. The sensor of claim 10 wherein the conductive coating material is silver.
